(19) United States
(12) Patent Application Publication
(10) Pub. No.: US 2005/0063926 A1
(43) Pub. Date: Mar. 24, 2005

(54) Film-forming compositions for protecting animal skin

(76) Inventors: Harinath B. Bathina, Lenexa, KS (US); Pablo M. Janowicz, Lenexa, KS (US); Thomas C. Hemling, Lake Winnebago, MO (US)

Correspondence Address: HOVEY WILLIAMS LLP, Suite 400, 2405 Grand Boulevard, Kansas City, MO, 64108, US

(21) Appl. No.: 10/668,640

(22) Filed: Sep. 23, 2003

(51) Int. Cl. A61K 7/04; A61K 7/06; A61K 7/11; A61K 9/14

(52) U.S. Cl. 424/61; 424/70.17; 424/70.13

(57) ABSTRACT

Improved film-forming skin protectant compositions are provided which are capable of forming a long-lasting elastic barrier film when applied to skin; the compositions have particular utility as barrier teat dips, for protecting cows against mastitis, especially during their susceptible non-lactating periods. The compositions include a film-forming component (preferably a mixture of polyether polyurethane and nitrocellulose) dispersed in a compatible carrier and further having a quantity of a tackifier resin incorporated therein in order to increase the time of adherence of the composition to skin, as compared with an otherwise identical composition without a tackifier resin. The compositions may also include a germicide (e.g., chlorhexidine diacetate), and a coloring dye.

### BACKGROUND OF THE INVENTION

1. Field of the Invention

The present invention is concerned with improved film-forming skin protectant compositions capable of forming an elastic film when applied to skin and including a film-forming component dispersed in a carrier, and wherein the compositions of the invention are improved by provision of a quantity of tackifier resin dispersed in the carrier; the tackifier resin serves to create a more long-lasting film capable of protecting skin (e.g., the skin of bovine teats) over relatively long periods of time. More particularly, the preferred forms of the invention pertain to film-forming compositions including therein effective amounts of polyether polyurethane and tackifier resin dispersed in tetrahydrafuran (THF) solvent, with a minor amount of nitrocellulose therein.

2. Description of the Prior Art

A significant problem for dairy farmers is the incidence of mastitis in cattle. It is known that up to 40-50% of inflammatory infections are contracted to a cow's dry or non-lactating period, with the greatest percentages of these infections occurring the first and last two weeks of the dry period. At these times, the mammary gland is in the transitional state where immunological factors are preoccupied or suppressed, milk is no longer being flushed from the gland, and increased mammary pressure distends the teat, thus allowing for easier bacterial penetration through the milk canal.

It is known to apply protective compositions to bovine teats, especially during or leading up to the non-lactating period, in order to minimize the occurrence of mastitis. The primary goal in such mastitis treatment is to minimize bacterial exposure on the teat ends.

U.S. Pat. No. 5,192,536 describes protectant compositions containing polyether polyurethane dissolved in tetrahydrafuran. The compositions of this patent can be applied to animal skin to form a rapidly drying, elastic film having protectant qualities. U.S. Pat. No. 5,688,498 describes further compositions of this general character, but which are improved by the provision of benzoin gum (benzoin resinoid). It has been found that the benzoin gum significantly increases the adhesion time of the protectant compositions and renders the compositions suitable for dipping so as to improve the coverage of the compositions and for ease of use.

U.S. Pat. No. 6,277,364 describes polyurethane compositions for the protection of skin from substances which are capable of penetrating the skin. U.S. Pat. No. 6,440,442 discloses a film-forming polymer blend comprising polyurethane and poly(N-vinyl lactam) for use during a mammal's dry period.

Furthermore, U.S. Pat. No. 6,030,633 (incorporated by reference herein) discloses compositions including a film-forming polyether polyurethane, benzoin gum, and a minor amount of nitrocellulose to increase the time of adherence of the composition to skin dispersed in a compatible carrier.

U.S. Pat. No. 5,942,239 is related to the '498 patent and describes further compositions making use of a variety of different solvents and additional ingredients such as germicides, dyes and optional ingredients, e.g., fillers, moisturizers, perfumes, and viscosity modifiers.

In addition to mastitis control, skin-protectant compositions also find utility as wound dressings to be applied to open wounds to protect against infection from dirt, insects or other sources of bacteria.

In order to enhance the protectiveness of skin protectant compositions of the forgoing types, it is desirable that it should remain intact on skin for as long as possible. This not only provides an added measure of protection against infection, but also lessens the time and expense involved in repeated reapplications of the compositions.

Tackifier resins are unique resinous materials generally characterized by low molecular weights. These resins are typically derived from petroleum or other organic, natural sources. Tackifier resins are especially known in the field of adhesives and have been studied for their functionality as such. Pocius, Alphonsus V., Adhesion and Adhesives Technology, Hanser Publishers, ISBN 1-56990-212-7, 1997.

There is accordingly a need in the art for improved skin-protectant compositions which maintain the ease of use and protection capabilities of the known polyether polyurethane/synthetic rubber/polyacrylic compositions while giving increased wear integrity and longevity.

### SUMMARY OF THE INVENTION

The present invention overcomes the problems outlined above, and provides greatly improved skin protectant compositions capable of forming an elastic film when applied to skin; such compositions broadly include a film-forming component comprising (preferably consisting essentially of) polyether polyurethane dispersed in a carrier wherein the improvement of the invention comprises the addition of a quantity of tackifier resin dispersed in the carrier in an amount sufficient to increase the time of adherence of the compositions to the skin, as compared with an otherwise identical composition which does not include the tackifier resin. Preferably, the film-forming component will also comprise a quantity of nitrocellulose.

In the simplest of terms, a tackifier resin is a resinous adhesive. For purposes of the present invention, preferred tackifier resins are materials other than gums and are selected from the group consisting of rosing ester resins, terpene resins, aliphatic and aromatic hydrocarbon resins, aliphatic-aromatic hydrocarbon resins, dimer ester resins, silane matrix resins, alkyl phenolic resins, and combinations thereof.

Rosin ester resins are derivatives of natural rosin material including gum rosin, wood rosin, and tall oil rosin (which are generated from the pine tree). Rosin can be hydrogenated to improve its stability, and further may be converted to a rosin ester using various alcohols. Rosin resins have a wide span of compatibility with a great number of polymers and are particularly well known for their peel and tack contribution to adhesives. Preferred rosin ester resins offer a narrow molecular weight distribution, light color, oxidation resistance, and superior chemical adhesion properties. Exemplary rosin ester resins include glycrin-rosin ester and pentaerythrytol rosin ester available from Hercules Chemical Co. Inc., Passaic, N.J., as Foral® 85 and 105, respectively, and the NovaRes® product line available from Georgia-Pacific.

Terpenes, generally, include any of many hydrocarbons or hydrocarbon derivatives that are found especially in plant oils, and produce many of the scents and tastes of plant products (such as pine needles). Terpene resins may be formed by the polymerization of the alpha and beta pinenes cationically using an aluminum chloride catalyst to increase the material softening point and molecular weight. Exemplary terpene resins for use with the present invention include the Piccolyte® series of polymers available from Hercules Chemical Co., the polyterpene Nirez available from Reichold, Inc., Durham, N.C., and Zonarez available from Arizona Chemical, Jacksonville, Fla.

A number of tackifier resins are petroleum based, and include aliphatic and aromatic hydrocarbon resins, aliphatic-aromatic hydrocarbon resins. There are three major types of petroleum based hydrocarbon tackifier resin materials: C5 aliphatic resins, C9 aromatic resins, and C5/C9 aliphatic-aromatic resins. Generally, feed streams to produce hydrocarbon resins can be divided into two groups: C5 piperylene feed stock and C9 resin oil. C5 piperylene feed stock contains various unsaturated C5 monomers such as pentenes and cyclopentenes. This C5 feed stock can be polymerized into a resin using a Lewis acid catalyst. Exemplary C5 resins include Excorez 1310 from Exxon, and Wingtack 10 from Goodyear.

C9 resin oil contains various monomers such as vinyltoluenes, dicyclopentadienes, indenes, and styrenes. A cationic polymerization reaction converts the liquid feedstock into a hard resin. C9 resins are aromatic molecules and are available in a wide variety of softening points and molecular weights.

C5/C9 aliphatic-aromatic resins are generally formed by mixing C5 feed stock and C9 resin oil streams. C5/C9 aliphatic-aromatic resins may also be referred to as aromatically modified C5 hydrocarbon resins. The ratio of the streams determines the aliphatic/aromatic balance of the resin and the degree of polymerization determines the molecular weight range and the physical properties of the resin. Exemplary C5/C9 aliphatic-aromatic resins include Excorez 2010 from Exxon, Piccovar from Hercules Chemical, and Goodyear's Wingtack Extra (which is actually a terpolymer containing piperylene, 2-methyl-2-butene, and alphamethylstyrene).

Preferred C5, C9 and C5/C9 hydrocarbon resins have molecular weights ranging from about 1300 to greater than 2000, aromaticity from about 0-30%, and softening temperatures of 85°-98° F. These properties are reflected in the difference in the solubility of the resin in the film-forming component and solvent system and differences in adhesiveness.

Dimerate polyol esters are preferred types of dimer ester resins which may be used with the present invention. The dimerate polyol esters preferably have molecular weights ranging from about 1300 to 3000 and are generally synthesized from dimer fatty acids. Such esters have in the past been used in polyurethane hot melt adhesives, aqueous dispersion adhesives, solvent born adhesives, and radiation curable adhesives. Exemplary dimerate polyol esters for use with the present invention include the Priplast series (3190-3198) available from Uniquema, New Castle, Del.

Other preferred tackifier resins for use with the present invention include capped hydrolyzed quadrafunctional silanes matrix (MQ) series and tertiary butyl phenolic resins.

In preferred embodiments, the tackifier resin is present at a level of about 1-20% by weight in the film-forming compositions according to the present invention, and more preferably at a level of about 1-10% by weight, and most preferably about 2-8% by weight. Virtually, any commercially available tackifier resin, particularly those preferred tackifier resins described above, are useful in the context of the present invention, although amounts of use for respective grades of tackifier resins may vary.

The preferred film-forming component of the present invention comprises polyether polyurethane and more preferably comprises a mixture of polyether polyurethane and nitrocellulose. The overall film-forming component is preferably present at a level of from about 5-50% by weight, and more preferably from about 7-25% by weight in the compositions of the invention. In more detail, the polyether polyurethane is normally used at a level of from about 5-25% by weight, more preferably from about 5-15% by weight; on the other hand, the nitrocellulose is normally present at a level of from about 0-20% by weight, and more preferably at a level of from about 1-10% by weight.

The carrier portion of the compositions is preferably a solvent for the film-forming component. The most preferred solvent is a mixture of tetrahydrafuran and alcohol, with the latter component being present at a level of from about 0.1-30% by weight, and more preferably at a level of from about 5-25% by weight. In addition to the foregoing though, a wide variety of other solvents can be used so long as the overall composition is stable at ambient temperature and has a sufficiently rapid drying time on the skin (preferably up to about 20 minutes, and more preferably up to about 10 minutes following skin application). Suitable solvents can therefore be selected from the group consisting of THF, cyclohexanone, toluene, acetone, the alkylene glycols (e.g., propylene or ethylene glycol) and the C₁-C₄ ethers, ketones and alcohols (trimethylether, diethylether, methylethylether, acetone, methylethylketone, diethylketone, methanol, ethanol, propanol, isopropanol or butanol).

The compositions of the invention may also have a germicidal agent dispersed in the carrier, which is normally used at a level of from about 0.01-2% by weight, and more preferably at a level of from about 0.05-1% by weight. The germicidal agent may be selected from a wide variety of effective agents, such as those taken from the group consisting of the linear or branched chain fatty acids (capric and caprylic acids, octanoic acid, nonanoic acid, decanoic acid), lactic acid, salicylic acid, bronopol, sodium pyridinathione, polyhexamethylene biguanide, chlorhexidine diacetate, dodecylbenzene sulfonic acid, and quaternary ammonium compounds such as the salts of alkyl dimethylbenzyl ammonium, dialkyl dimethyl ammonium compounds and benzthonium. Combinations of such germicides may also be used, so long as the germicides themselves, and in combination with the other ingredients, form compatible compositions which are physically and chemically stable at ambient temperatures.

In preferred embodiments, various adhesion promoters (also referred to as skin penetrants and surfactants) are added to the film forming composition. Preferred adhesion promoters include but are not limited to polyvinylpyrrolidone, polyethylene glycol, and polyethylene polypropylene glycol (poloxamer).

Dyes may also be added to the skin protectant compositions of the invention in order to improve the visualization of the barrier film on the skin area being treated. Acceptable dyes include those that are soluble or dispersible in the liquid compositions of the invention. The preferred dyes provide easy visualization of the barrier film from a distance of at least 20 feet and can be used at relatively low concentrations. Dyes should be used at a level of from about 0.001-0.5% by weight in the compositions, and more preferably at a level of from about 0.005-0.2% by weight. Dyes are normally selected from the group consisting of the D&C red 19, solvent yellow 43, fluoro yellow napthalic acid amide, Keyplast yellow TGH, FD&C red 3, FD&C red 22, FD&C red 28, FD&C red 39, FD&C red 19, FD&C green 6, orange 5, orange 10, orange 17, red 17, red 21, red 27, red 31, violet 2, yellow 7 and yellow 11.

In order to be most useful the compositions of the invention should have a desirable viscosity measured at 25° C. using a Brookfield viscometer, spindle #2 at 30 rpm should be at a level of from about 50-5000 cPs, more preferably from about 100-2000 cPs. As indicated previously, the compositions should have a skin drying time at about 25° C. (ambient temperature) of up to about 20 minutes and more preferably up to about 10 minutes, the drying time being determined by the time for a 5 gram sample poured onto a Petri dish to become dry to the touch at 25° C.

Preferably, compositions according to the present invention have retention times on the animal skin (preferably bovine teats) at least about 10% greater as compared with an otherwise identical composition including only benzoin gum and no other tackifier resin. More preferably, the percent improvement in retention time is at least about 15%, and most preferably at least about 30%.

The composition is used in the manner of typical teat dip products, i.e., the bovine teats are dipped into liquid compositions in order to create the long-lasting protectant films of the invention.

However, if desired, the compositions hereof can be applied by other means, such as by wiping or spraying so as to create elastic films which are vapor permeable, water-, wind-, dirt-, insect-proof and present a barrier to bacteria. The film protects the skin from damage or exposure during the healing process (in the case of wound treatments) or entrance of mastitis-causing bacteria when used as a teat dip.

### DETAILED DESCRIPTION OF THE PREFERRED EMBODIMENT

The following examples illustrate preferred film protectant compositions in accordance with the invention, and demonstrate the long-lasting nature of the elastic films using such compositions. It is to be understood however, that the examples are provided by way of illustration only, and nothing therein should be taken as a limitation upon the overall scope of the invention.

### EXAMPLES

In each of the following examples, the compositions were prepared by dissolving the polyether polyurethane/tackifier resin film-forming component in a THF and ethanol solvent system with subsequent addition of nitrocellulose and other ingredients. All mixing was carried out at ambient conditions until a clear solution was obtained. Care was taken to prevent excessive evaporation of solvent. The exemplary compositions are summarized in Table 1.

TABLE 1

| Ingredient | | | | | | |
|---|---|---|---|---|---|---|
| (parts by wt.) | Example 1 | Example 2 | Example 3 | Example 4 | Example 5 | Example 6 |
| Tetrahydrofuran | 76.85 | 76.85 | 76.90 | 76.65 | 75.90 | 76.85 |
| Estane 5714 | 10.35 | 10.35 | 9.00 | 10.35 | 10.50 | 10.35 |
| Ethanol | 5.00 | 5.00 | 5.00 | 5.00 | 5.00 | 5.00 |
| D & C red dye #28 | 0.10 | 0.10 | 0.10 | 0.10 | 0.10 | 0.10 |
| Nitrocellulose R-15 | 1.50 | 1.50 | — | 1.50 | 1.50 | 1.50 |
| Benzoin gum | — | — | — | — | — | 1.20 |
| Wingtack Extra¹ | 6.20 | — | — | 6.210 | 6.00 | 5.00 |
| NovaRes 1100² | — | 6.20 | 6.00 | — | — | — |
| Priplast 3192³ | — | — | — | — | — | — |
| PVP 904 | — | — | 3.00 | — | — | — |
| Capric/caprylic acid | — | — | — | 0.20 | — | — |
| Polyethylene glycol | — | — | — | — | 1.00 | — |
| (approx. MW = 400) | | | | | | |

¹Petroleum-based, C5 hydrocarbon tackifier resin from Goodyear Chemical.
²Tall oil rosin and modified ester tackifier resin from Geogia-Pacific.
³Dimerate polyol ester tackifier resin from Uniqema.

Examples 1-2, the nitrocellulose was added to and mixed with the quantity of THF and then, while mixing, adding the nitrocellulose. The tackifier resin (Wingtack, NovaRes, and Priplast, respectively) was added the composition mixed. The film-forming ingredient, Estane, was added and the composition mixed for 2-4 hours until the resin dissolved. The dye and ethanol were mixed into the resin composition.

In Example 3, the PVP was mixed with the THF, followed by the addition of the tackifier resin NovaRes. The film-forming ingredient Estane was added and the composition mixed for 2-4 hours until the resin dissolved. Finally, the dye and ethanol were added and mixed into the composition.

In Example 4, the nitrocellulose was mixed with the THF, followed by the addition of the germicidal agent, capric/caprylic acid. The tackifier resin was added and the composition mixed. The quantity of Estane was then added and the composition mixed for 2-4 hours until the resin dissolved. Lastly, the dye and ethanol were added and mixed into the composition.

In Example 5, the nitrocellulose was mixed with the THF, followed by the addition of the polyethylene glycol. The tackifier resin Wingtack was added to the composition and mixed. The Estane was added and the composition mixed for 2-4 hours until the resin dissolved. Finally, the quantities of dye and ethanol were added.

Example 6 was prepared by mixing the nitrocellulose with THF, followed by the addition of the benzoin gum. The tackifier resin Wingtack was then added and the composition mixed. Next, Estane was added and the composition mixed for 2-4 hours until the resin dissolved. Dye and ethanol were then added and the composition mixed.

The adherence time of Examples 1-6 were then compared with a composition prepared as disclosed in U.S. Pat. No. 6,030,633 (trial P of Table 8), without any tackifier resin. The compositions were applied to the teats of cows in order to determine the period of time the teat would be protected from bacteria. All gross contaminants were removed from the animal using a paper or cloth towel. The animals were then given a standard disinfection with a predip low in emollients. The teat skin, upon being clean and dry, was treated with a 70% isopropyl alcohol solution. Using a teat applicator cup, the teats were immersed to the base of the udder in the experimental composition. The cows were allowed to stand until the product dried. In total, five trials were performed, the results of which are summarized in Table 2.

TABLE 2

| | Days Test at Least 50% of Cows Protected | | | | | | |
|---|---|---|---|---|---|---|---|
| Trial | Control | Example 1 | Example 2 | Example 3 | Example 4 | Example 5 | Example 6 |
| 1 | 3.25 | 3.75 | — | — | — | — | — |
| 2 | 4 | — | 7 | — | — | — | — |
| 3 | 3.5 | — | >5 | 45.5 | — | — | — |
| 4 | 3.75 | 5.33 | — | — | 5.33 | — | 5 |
| 5 | 4 | 5.5 | — | — | — | 4.37 | — |
| Avg. % | — | 31% | 89% | 22% | 44% | 18% | 35% |
| Viscosity (cPs) | 284 | 1020 | — | — | — | 2288 | — |

All of the compositions tested showed increased protection times when compared with the control formulation without any tackifier resin. Variations among the results for each particular example result from variations in trial conditions such as the size and type of cow (and teat), the weather, and preparation of the teat. Preferably, compositions according to the present invention exhibit retention times of at least about 10% over the control, more preferably at least about 15%, and most preferably at least about 30%.

A wide variety of specific ingredients can be used in the formulations of the invention and at varying usage levels. Table 3 sets forth approximate broad and preferred ranges for essential and preferred ingredients used in the formulations, and also sets forth broad and preferred viscosity ranges.

TABLE 3

| | | |
|---|---|---|
| | Approximate Broad | Approximate Pre- |
| Ingredient | Range | ferred Range |

Essential Ingredients (% by weight)

| | | |
|---|---|---|
| Film-forming component | 5-50 | 7-25 |
| Tackifier resin | 1-20 | 1-10 |
| Carrier | qs | qs |

Preferred Ingredients (% by weight)

| | | |
|---|---|---|
| Nitrocellulose | 0-20 | 1-10 |
| Polyether polyurethane | 5-25 | 5-15 |
| Benzoin gum | 0-20 | 0-10 |
| Adhesion promoter/penetrant | 0-20 | 0-10 |
| Germicide | 0.01-2 | 0.05-1 |
| Dye | 0.001-0.5 | 0.005-0.22 |
| Alcohol | 0-30 | 5-20 |
| THF | qs | qs |
| Viscosity (cPs) | 50-5000 | 100-2000 |

1. In a film-forming skin protectant composition capable of forming an elastic film when applied to skin and including a film-forming component comprising polyether polyurethane dispersed in a carrier, the improvement which comprises a quantity of tackifier resin dispersed therein, said tackifier resin being a resinous material other than a gum.

2. The composition of claim 1, said tackifier resin being selected from the group consisting of rosin ester resins, terpene resins, aliphatic and aromatic hydrocarbon resins, aliphatic-aromatic hydrocarbon resins, dimer ester resins, silane matrix resins, alkyl phenolic resins, and combinations thereof.

3. The composition of claim 2, said tackifier resin selected from the group consisting of tall oil rosin esters, glycerin rosin esters, pentaerythrytol rosin esters, polyterpene resins, aliphatic C5 resins, C5/C9 aliphatic-aromatic, dimerate polyol ester resins, capped hydrolyzed quadrafunctional silane matrix resins, tertiary butyl phenolic resins, and combinations thereof.

4. The composition of claim 1, said tackifier resin being present in the composition at a level of from about 1-20% by weight.

5. The composition of claim 1, said film-forming component further comprising a quantity of nitrocellulose.

6. The composition of claim 1, said film-forming component further comprising a quantity of benzoin gum.

7. The composition of claim 1, said polyether polyurethane being present at a level of from about 5-25% by weight in the composition.

8. The composition of claim 1, said carrier being a solvent for said film-forming component and selected from the group consisting of THF, cyclohexane, toluene, ethers, ketones, alcohols, alkylene glycols and mixtures thereof.

9. The composition of claim 1, including a germicidal agent dispersed in said carrier.

10. The composition of claim 9, said germicidal agent selected from the group consisting of linear or branched chain fatty acids, bronopol, sodium pyridinathione, poly-hexamethylene biguanide, chlorhexidine diacetate, quaternary ammonium compounds and mixtures thereof.

11. The composition of claim 1, said composition having a viscosity of from about 50-5000 cPs.

12. A film-forming composition for protecting animal skin comprising:
from about 5-50% by weight of a film-forming component consisting essentially of a mixture of polyether polyurethane and nitrocellulose; and
from about 1-20% by weight of a tackifier resin,
said film-forming component and said tackifier resin being dispersed in a carrier.

13. The composition of claim 12, said carrier being a solvent for said film-forming component and selected from the group consisting of THF, cyclohexane, toluene, ethers, ketones, alcohols, alkylene glycols, and mixtures thereof.

14. The composition of claim 12, said composition comprising from about 5-25% by weight polyether polyurethane and from about 0.2-20% by weight nitrocellulose.

15. The composition of claim 12, said composition further comprising up to about 20% by weight benzoin gum.

16. The composition of claim 12, said tackifier resin being selected from the group consisting of rosin ester resins, terpene resins, aliphatic and aromatic hydrocarbon resins, aliphatic-aromatic hydrocarbon resins, dimer ester resins, silane matrix resins, alkyl phenolic resins, and combinations thereof.

17. The composition of claim 16, said tackifier resin selected from the group consisting of tall oil rosin esters, glycerin rosin esters, pentaerythrytol rosin esters, polyterpene resins, aliphatic C5 resins, C5/C9 aliphatic-aromatic resins, dimerate polyol ester resins, capped hydrolyzed quadrafunctional silane matrix resins, tertiary butyl phenolic resins, and combinations thereof.

18. The composition of claim 12, said composition further comprising a germicidal agent.

19. The composition of claim 18, said germicidal agent selected from the group consisting of linear or branched chain fatty acids, bronopol, sodium pyridinathione, poly-hexamethylene biguanide, chlorhexidine diacetate, quaternary ammonium compounds and mixtures thereof.

20. The composition of claim 12, said composition having a viscosity of from about 50-5000 cPs.

21. A film-forming composition for protecting bovine teats comprising:
from about 5-50% by weight of a film-forming component comprising polyether polyurethane; and
from about 1-20% by weight of a tackifier resin,
said film-forming component and said tackifier resin being dispersed in a carrier,
said composition having a retention time at least about 10% greater as compared with an otherwise identical composition including only benzoin gum and no other tackifier resin.

22. The composition of claim 21, said retention time being at least about 15% greater as compared with said otherwise identical composition.

23. The composition of claim 21, said tackifier resin being selected from the group consisting of rosin ester resins, terpene resins, aliphatic and aromatic hydrocarbon resins, aliphatic-aromatic hydrocarbon resins, dimer ester resins, silane matrix resins, alkyl phenolic resins, and combinations thereof.

24. The composition of claim 23, said tackifier resin selected from the group consisting of tall oil rosin esters, glycerin rosin esters, pentaerythrytol rosin esters, polyterpene resins, aliphatic C5 resins, C5/C9 aliphatic-aromatic resins, dimerate polyol ester resins, capped hydrolyzed quadrafunctional silane matrix resins, tertiary butyl phenolic resins, and combinations thereof.

25. The composition of claim 21, said film-forming component further comprising a quantity of nitrocellulose.

26. The composition of claim 21, said film-forming component further comprising a quantity of benzoin gum.

27. The composition of claim 21, said polyether polyurethane being present at a level of from about 5-25% by weight in the composition.

28. The composition of claim 21, said carrier being a solvent for said film-forming component and selected from the group consisting of THF, cyclohexane, toluene, ethers, ketones, alcohols, alkylene glycols and mixtures thereof.

29. The composition of claim 21, including a germicidal agent dispersed in said carrier.

30. The composition of claim 29, said germicidal agent selected from the group consisting of linear or branched chain fatty acids, bronopol, sodium pyridinathione, poly-hexamethylene biguanide, chlorhexidine diacetate, quaternary ammonium compounds and mixtures thereof.

31. The composition of claim 46, including a dye dispersed in said carrier.

32. The composition of claim 46, said composition having a viscosity of from about 50-5000 cPs.

33. A method of protecting bovine teats comprising the step of applying to such teats the composition of claim 1, and allowing the applied composition to dry thereon and form an elastic, teat-protecting film.

34. The method of claim 33, said teats being the teats of non-lactating cows.

35. A method of protecting bovine teats comprising the step of applying to such teats the composition of claim 12, and allowing the applied composition to dry thereon and form an elastic, teat-protecting film.

36. The method of claim 35, said teats being teats of non-lactating cows.

37. A method of protecting bovine teats comprising the step of applying to such teats the composition of claim 21, and allowing the applied composition to dry thereon and form an elastic, teat-protecting film.

38. The method of claim 37, said teats being teats of non-lactating cows.
